(19) United States
(12) Patent Application Publication
(10) Pub. No.: US 2025/0319118 A1
(43) Pub. Date: Oct. 16, 2025

(54) Prophylaxis and Treatment of Degraded Cartilage

(71) Applicant: PhrixionLS, LLC, Wilmington, DE (US)

(72) Inventors: Jens STABLER, Wilmington, DE (US); Courtney ZIEGLER, Wilmington, DE (US); Scott MCCLURE, Wilmington, DE (US); Kalle Levon, Wilmington, DE (US)

(21) Appl. No.: 18/871,889

(22) PCT Filed: Jun. 6, 2023
(86) PCT No.: PCT/US2023/024540

§ 371 Date: Dec. 5, 2024

Related U.S. Application Data
(60) Provisional application No. 63/349,341, filed on Jun. 6, 2022.

(51) Int. Cl. A61K 31/785; A61K 33/38; A61K 45/06

(52) U.S. Cl. CPC A61K 31/785; A61K 33/38; A61K 45/06

(57) ABSTRACT

The present invention relates to the use of certain polyacrylamide gel formulations to treat conditions where cartilage has degraded and/or where exposed collagen is present. The invention further relates to the prophylactic use of such formulations to prevent degradation, or further degradation, of cartilage and/or collagen. Additionally, the invention also includes substituting such certain polyacrylamide gel formulations in lieu of carboxymethylcellulose, or alternatively HA, in individuals and mammals having allergies, sensitivities, or other adverse reactions to such compounds. Additionally, the invention further relates to the use of the polyacrylamide gel formulations for the controlled release of therapeutic agents and/or as a medical scaffold or matrix for biological therapies.

### PRIORITY CLAIM AND CROSS-REFERENCE TO RELATED APPLICATIONS

This application claims the benefit of U.S. Provisional Patent Application No. 63/349,341, filed Jun. 6, 2023.

### FIELD OF TECHNOLOGY

The present invention relates to the use of certain polyacrylamide gel formulations to treat conditions where cartilage has degraded and/or where exposed collagen is present. The invention further relates to the prophylactic use of such formulations to prevent degradation, or further degradation, of cartilage and/or collagen. Additionally, the invention also includes substituting such certain polyacrylamide gel formulations in lieu of carboxymethylcellulose, or alternatively HA, in individuals and mammals having allergies, sensitivities, or other adverse reactions to such compounds. Additionally, the invention further relates to the use of the polyacrylamide gel formulations for the controlled release of therapeutic agents and/or as a medical scaffold or matrix for biological therapies.

### BACKGROUND

### Cartilage Damage: Repair and Prevention

Cartilage is a connective tissue found throughout the body of both humans and mammals. When intact, cartilage both contributes to skeletal and muscular structure and carries out important functions necessary for movement. All cartilage, i.e., hyaline, fibrous, and elastic cartilage, is made of chondrocytes or specialized cartilage cells that produce a number of proteins, including collagen fibers and elastic fibers that provide cartilage with flexibility and strength. Having a smooth surface, healthy cartilage also is the body's shock absorber as well as minimizing friction between surfaces and facilitating ease of motion.

Certain diseases, as well as repetitive motions, injuries, and the gradual wear and tear associated with aging, can damage cartilage. Damaged cartilage may contain cracks and fissures in the smooth surface, exposing collagen. Damaged cartilage causes a variety of physical symptoms, including inflammation, stiffness, grinding or clicking in the joint during movement, and, of course, pain.

Current treatments for damaged cartilage are typically invasive. As one example, a surgeon may drill holes in the bone or bone marrow under the damaged cartilage, which prompts the development of fibrocartilage. In other procedures, cartilage is transplanted—from healthy sites to the damaged portions or from cadaver tissues. Cartilage cells also may be harvested, cloned, grown, and then implanted in the damaged site, a process that takes several weeks. Stem cell therapies are also under investigation.

Unfortunately, these more invasive treatment options have not established sufficient outcomes to be considered an “ideal cure” for articular cartilage damage. Fibrocartilage is an inferior tissue than healthy hyaline cartilage, and therefore also deteriorates with time. Cartilage transplant procedures are limited to use with i) a stable knee with intact cruciate ligaments, ii) a straight joint axis without deformity, iii) cases with no loose bodies (small fragments of cartilage or bone), iv) preserved menisci, and v) cartilage only defect on one of the two joint surfaces. Cartilage transplantation is further an elaborate process and very sensitive to the introduction of infectious organisms, as the cells require harvesting, growth in a lab under sterile conditions for several weeks, and then transportation of the cell aggregates back to the hospital or clinic for transplantation. Stem cell therapies also require harvesting and growth in a lab, carrying similar difficulties and risks as with cartilage transplantation. Additional challenges with stem cell therapy include relatively low cell growth rate, reduced differentiation capacity for different tissues (source dependent), and inadequate effects in clinical trials. See Zhu, C., Wu, W., & Qu, X., Mesenchymal stem cells in osteoarthritis therapy: a review, 13(2) AM. J. TRANSLATIONAL RES. 448-461 (2021); Sofu, H., et al., Lesion size and varus malalignment are the major determinants leading to poorer clinical outcomes after combined microfracture treatment for focal cartilage lesions during anterior cruciate ligament reconstruction, 142(8) ARCHIVES ORTHOPAEDIC & TRAUMA SURGERY 1941-1949 (2022).

Most treatments for damaged cartilage address the symptoms associated with cartilage degradation, i.e., they reduce inflammation, provide an outside lubrication source such as hyaluronic acid (“HA”) (i.e., viscosupplementation), and/or target pain. Current treatments further provide limited and short-term relief. Viscosupplementation with HA, specifically, has received mixed reviews. HA is susceptible to enzymatic degradation and has a short lifespan following injection. Reactions to HA also have been observed, including inflammation reactions, limiting the ability to use HA in certain humans and animals. Some analyses of the use of HA in viscosupplementation even indicate that HA offers no significant benefit compared to placebo. See, e.g., Balazs E A, Delinger J L, Viscosupplementation: A new concept in the treatment of osteoarthritis, 20(39) J. OF RHEUMATOLOGY 3-9 (1993); Bellamy N, Campbell J, Robinson V, et al., Viscosupplementation for the treatment of osteoarthritis of the knee, 2 COCHRANE DATABASE SYST. REV. CD005321 (2006).

There are also few options for preventing the damage of cartilage. Prophylactic treatments currently focus on obtaining a healthy weight, exercising regularly, and avoiding injury. Prophylactic treatments for ostcoarthritis, as one example, often consist of the use of nutraceuticals or drugs that contain substances that are building-blocks for cartilage, in order to improve the availability of these substances in the body to heal microdamage before tissue degeneration progresses. Data on whether such products, such as glucosamine and chondroitin sulfate, are used by the body effectively to rebuild cartilage is inconclusive, however. See, e.g., Mithoefer, K., et al., “New Frontiers for Cartilage Repair and Protection”, 3(1) CARTILAGE 77S-86S (January 2012).

Disease modifying osteoarthritis drugs (DMOADs) are intended to delay or halt the progression of osteoarthritis and cartilage degeneration. It has also been proposed that these drugs could be used to prevent the development of osteoarthritis entirely. For drugs to be eligible for this classification, they must positively alter disease progression and symptoms as defined by the United States FDA. There are currently no products that have been approved by the FDA as a DMOAD. Investigational DMOAD development, addressing cartilage degeneration, has included proteinase inhibitors, fibroblast growth factor, Wnt signaling inhibitors, transforming growth factor-β, and senolytic therapies. These investigational products attempt to address catabolic and reparative mechanisms at a molecular level. Challenges associated with these products have included musculoskeletal toxicities, and a lack of efficacy in preventing disease progression and/or controlling symptoms of osteoarthritis including pain and joint function. See Oo, W. M., et al., The Development of Disease-Modifying Therapies for Osteoarthritis (DMOADs): The Evidence to Date, 15 DRUG DESIGN, DEVELOPMENT & THERAPY 2921-2945 (2021).

Below is a summary of exemplary dietary supplements and drugs that have been investigated as prophylactic treatment for osteoarthritis or disease-modifiers for cartilage damage. The limitations of each supplement and drug are set forth below in Chart 1.

CHART 1

| Product | Category | Limitations | Reference |
|---|---|---|---|
| Glucosamine | Dietary | Inconsistent outcomes. Clinical | https://www.ncbi.nlm.nih.gov/pmc/articles/PMC5187842/ |
| and/or | Supplement | and preclinical data suggest | |
| Chondroitin | | stimulation of cartilage | |
| Sulfate | | regeneration, however, in | |
| | | clinical trials, a large number | |
| | | of individuals do not benefit | |
| | | from treatment. | |
| Fish Oil | Dietary | Lack of human clinical trials | https://www.ncbi.nlm.nih.gov/pmc/articles/PMC5187842/ |
| | Supplement | showing effectiveness. Ideal | https://ard.bmj.com/content/75/4/e13 |
| | | dose is unknown, and has been | |
| | | observed to increase risk of | |
| | | infection, cancer, | |
| | | gastrointestinal events, and | |
| | | cardiovascular disease. | |
| Hyaluronic | Dietary | No long-term results available. | https://www.ncbi.nlm.nih.gov/pmc/articles/PMC5187842/ |
| Acid | Supplement | | |
| S-adenosyl- | Dietary | Large dosing ranges for effect. | https://www.ncbi.nlm.nih.gov/pmc/articles/PMC5187842/ |
| methionine | Supplement | Shown to improve OA symptoms, | |
| (SAMe) | | unknown if its use helps with | |
| | | disease progression long term. | |
| Collagen | Dietary | Shown to improve OA symptoms, | https://www.ncbi.nlm.nih.gov/pmc/articles/PMC5187842/ |
| (UC-II) | Supplement | unknown if its use helps with | |
| | | disease progression long term. | |
| Avocado/soy | Dietary | Shown to improve OA symptoms, | https://www.ncbi.nlm.nih.gov/pmc/articles/PMC5187842/ |
| unsaponifiable | Supplement | unknown if its use helps with | |
| | | disease progression long term. | |
| Boswellia | Dietary | Shown to improve OA symptoms, | https://www.ncbi.nlm.nih.gov/pmc/articles/PMC5187842/ |
| | Supplement | unknown if its use helps with | |
| | | disease progression long term. | |
| Phytoflavonoids | Dietary | Shown to improve OA symptoms, | https://www.ncbi.nlm.nih.gov/pmc/articles/PMC5187842/ |
| and | Supplement | unknown if its use helps with | |
| Bioflavonoids | | disease progression long term. | |
| Flavocoxid | Dietary | Shown to improve OA symptoms, | https://onlinelibrary.wiley.com/doi/full/10.1016/ |
| | Supplement | unknown if its use helps with | j.pmrj.2012.02.023?saml_referrer |
| | | disease progression long term. | |
| Devil's Claw | Dietary | Shown to improve OA symptoms, | https://www.ncbi.nlm.nih.gov/pmc/articles/PMC5187842/ |
| | Supplement | unknown if its use helps with | |
| | | disease progression long term. | |
| Bromelain | Dietary | Mixed results in clinical trials, | https://www.ncbi.nlm.nih.gov/pmc/articles/PMC5187842/ |
| | Supplement | requiring more rigorous trials. | |
| Ginger | Dietary | Shown to improve OA symptoms, | https://www.ncbi.nlm.nih.gov/pmc/articles/PMC5187842/ |
| | Supplement | unknown if its use helps with | |
| | | disease progression long term. | |
| GLPG1972 | Drug | No significant difference versus | https://www.dovepress.com/the-development-of-disease-modifying- |
| | (Trial) | placebo in Phase-2 study (efficacy). | therapies-for-osteoarthritis-dmoa-peer-reviewed-fulltext-article-DDDT |
| Sprifermin | Drug | Demonstrated positive effects | https://www.dovepress.com/the-development-of-disease-modifying- |
| | (Trial) | towards cartilage protection, | therapies-for-osteoarthritis-dmoa-peer-reviewed-fulltext-article-DDDT |
| | | but has varied in its ability | |
| | | to meet minimum threshold of | |
| | | improvement in pain. | |
| LNA043 | Drug | Efficacy trials ongoing. | https://ard.bmj.com/content/80/Suppl_1/363.2.abstract |
| | (Trial) | Initial results demonstrate | |
| | | potential to regenerate some | |
| | | articular cartilage lesions. | |
| TissueGene-C | Drug | Efficacy trials ongoing. | https://www.dovepress.com/the-development-of-disease-modifying- |
| | (Trial) | Initial trials demonstrated no | therapies-for-osteoarthritis-dmoa-peer-reviewed-fulltext-article-DDDT |
| | | significant effects towards | https://journals.sagepub.com/doi/full/10.1177/1759720X221090297 |
| | | cartilage protection. | |
| Lorecivivint | Drug | Efficacy trials ongoing. | https://www.dovepress.com/the-development-of-disease-modifying- |
| | (Trial) | | therapies-for-osteoarthritis-dmoa-peer-reviewed-fulltext-article-DDDT |
| UBX0101 | Drug | Efficacy trials ongoing | https://www.dovepress.com/the-development-of-disease-modifying- |
| | (Trial) | | therapies-for-osteoarthritis-dmoa-peer-reviewed-fulltext-article-DDDT |
| Fisetin | Drug | Efficacy trials ongoing | https://www.dovepress.com/the-development-of-disease-modifying- |
| | (Trial) | | therapies-for-osteoarthritis-dmoa-peer-reviewed-fulltext-article-DDDT |

There is a need for more effective treatments for cartilage damage and in particular non-invasive treatments that provide longer term relief. There further is a need for prophylactic treatments to prevent damage to healthy cartilage as well as help prevent further damage when degradation is already present.

### Exposed Collagen: Repair and Prevention of Adhesions

As is known in the art, damaged cartilage exposes, inter alia, collagen fibers. A number of conditions and disease states are thought to result from, or be exacerbated by, exposed collagen.

### Corneal Ulcers

For example, corneal ulcers (ulcerative keratitis) occur across mammals, occurring from direct trauma or irritation, as well as from some viral, bacterial, and fungal infections. Among other effects, degradation of the corneal collagen is observed with corneal ulcers. Ulcerations can range from superficial to deep and lead to perforation of the cornea with prolapse of the iris. This condition is painful, and may lead to loss of vision, and possibly loss of the eye. Secondary bacterial and fungal infections can worsen the condition and prolong the recovery period.

Treatment for corneal ulcers may vary significantly, as differing etiologies may exist and the development of secondary conditions may prevent healing. In addition to topical analgesic medications to control pain, cyclopegic drugs to control reflex uvcitis, antibiotics or antifungal medications as required, and amniotic and/or blood-based therapies to facilitate corneal wound healing, physical protection of the cornea remains an important consideration in managing this condition. Exposure keratopathy, where the cornea has prolonged exposure to the outside environment, can both cause and worsen corneal ulcers.

In humans, physical barriers that are currently utilized includes bandage contact lenses, biologic and nonbiologic glues, temporarily suturing the eyelid closed, and lubricating eye drops or gels. Bandage contact lenses are left on for two weeks to one month's time, with potential complications including infectious keratitis, dry eye, corneal hypoxia, and corneal edema. Biologic glues form a biologic sealant from fibrin and thrombin. This method is less toxic than non-biologic glues, although it is not readily available and may require multiple placements by a doctor during the healing process. Non-biologic glues are long-lasting, providing physical protection to the ulceration for upwards of 45 days. This is considered an off-label use, and requires careful placement by a doctor to ensure no glue enters the anterior chamber or spreads onto the ocular surface. Crust formation of the glue can also be irritating to the patient and scarring is expected underneath and adjacent to the glue. In the case of both biologic and non-biologic glue use, a bandage contact lens may still be required, and still requires frequent administration of antibiotics and anti-inflammatoires. Closing the eye by the placement of temporary sutures provides better results than utilizing an eye patch, and is recommended to perform this procedure early-on in cases not responding to medical therapies. Drawbacks of this treatment includes patient discomfort and cosmesis, and difficulty in examination of the ocular surface by doctors. Lubricating eye drops or gel protect the ocular surface and decrease friction of the eyelids over the cornea. Cellulose derivatives are often used, the most common being carboxymethylcellulose, which can cause exposure allergies in some individuals. Carboxymethylcellulose is a derivative of the plant polysaccharide cellulose, and has been documented to cause anaphylaxis in some individuals. As the product is used widely in food, detergents, drinks, and other everyday products, allergies can develop with repeat exposure causing anaphylaxis when administered as a medical treatment.

Lubricants with lanolin causes irritation and delays healing of the cornea. Retention time of these lubricants on the ocular surface varies, with one study finding a range of 10-minutes up to 90-minutes duration. See, e.g., Dang, D. H., Riaz, K. M., & Karamichos, D., Treatment of Non-Infectious Corneal Injury: Review of Diagnostic Agents, Therapeutic Medications, and Future Targets, 82(2) DRUGS 145-167 (2022); Araújo, D. M. L. de, & Galera, P. D., Ocular lubricants: what is the best choice? 46(11) CIÊNCIA RURAL 2055-2063 (2016), available at https://doi.org/10.1590/0103-8478cr20160020; Bedos, L., et al., Precorneal retention time of ocular lubricants measured with fluorophotometry in healthy dogs, VETERINARY OPHTHALMOLOGY (2023).

There is a need for additional mechanical barriers that can localize to the area of damaged tissue and help allow for re-epithelialization of the cornea to occur underneath. Historically, polyacrylamide has been added to other hydrogel preparations, in order to improve their characteristics for the purpose of drug delivery systems. A polyacrylamide hydrogel thus should not interact with common ophthalmic medications used for the treatment of corneal ulcers, including antibiotics, atropine, and anti-inflammatories.

### Wound Treatment

Additionally, collagen exposure is also associated with pressure ulcers, burn wounds, traumatic partial to full thickness injuries, and surgical partial to full thickness wounds. In many of such wounds, the lesion and/or wound must heal by second intention through granulation and scar tissue formation as opposed to surgical closure. Current treatments for such lesions and wounds include the use of hydrogel wound ointments that provide a moist healing environment for wound healing.

There is a need, however, for additional and superior protective barriers to help provide a separation between the wound and air. The ability to specifically target wounded tissue, remain localized to that tissue, and provide long-lasting coverage is also needed. There further is a need for such layers to be combined with slow release of therapeutics to prevent infection and aid in the healing process.

### Prevention of Tendon Adhesions

A further example of a condition related to exposed collagen is the development of tissue adhesions, such as in tendonitis and tendon fiber disruption. Tendons can be injured when they are overloaded or sustain direct trauma. Tendons often take a long time to heal and can heal with tissue adhesions to surrounding tissues in both humans and animals. This leads to morbidity post healing including loss of function, range of motion, etc. Current treatments for preventing tendon adhesions is varied. A device has been developed in human medicine, that is surgically placed on the tendon to provide lubrication between the tendon and adjacent tissue. Various surgical techniques in human healthcare also have been studied to decrease development of adhesions including the use of porcine acellular dermal matrix wrap and different suture techniques. In addition, the use of pharmacologics to control inflammation, physical therapy to maintain mobility and gliding of tissues, and hyaluronic acid injections are also utilized. There is a need for further non-invasive prevention treatments, however, to prevent tissue adhesions, including a need for treatments that can affix to the site of the injury, allow the damaged areas to heal, and provide long-lasting lubrication effects.

### Abrasive Damage: Repair and Prevention

In addition to cartilage, other structures help facilitate the glide of tissues in the body. These include bursas, including but not limited to synovial bursas, as well as tendon sheaths.

Bursas are fluid filled sacs that facilitate the gliding of various soft tissue structures over bone. Synovial bursas are components of the synovial joints that are filled with synovial fluid that provides a cushion between bones and tendons and/or muscles around a joint. During surgery on synovial joints, the synovial fluid is diluted and flushed from the joint, requiring the body to produce replacement fluid. Due to inflammation from surgery, the production of the fluid can be inferior to that of a non-inflamed joint, leading to osteoarthritis. In cases of joint reconstruction, inadequate lubrication may lead to wear of implants. Today, there are replacements for lubricating products that are used as treatments; they are applied into the joint to decrease friction. These products however are short-acting and subject to enzymatic degradation. A solution for longer-term lubrication after surgical intervention in a synovial joint is still needed.

### Bioform PAAGs

A product known as Noltrex Vet® (available through RC Bioform), is currently used to treat join pain in animals and, in some countries, in humans as a replacement for synovial fluid. Noltrex Vet® is a 3-5 wt % solution of crosslinked polyacrylamide hydrogel with a high molecular weight of approximately 10×10⁶ Dalton. It is comprised of 3-dimensional polyacrylamide, purified water, silver ions, and a phosphate buffer with a repeating unit of [—CH₂—CH(CONH₂)—]ₙ.

In addition to the Noltrex Vet® polyacrylamide hydrogel product, Bioform International SA owns U.S. Pat. No. 7,294,348, which has expired. The '348 patent describes and claims similarly matrixed and formulated polyacrylamide hydrogels and methods of their production. The U.S. Pat. No. 7,294,348 is incorporated herein by reference.

In this application, the term “Bioform PAAGs” or “bPAAGs” will be used to refer to the following: the Noltrex Vet® product as characterized above; a modification of the Noltrex Vet® product where silver ions are omitted; a modification of the Noltrex Vet® product where the phosphate buffer is omitted; a medication of the Noltrex Vet® product where silver ions and the phosphate buffer are omitted; a 2-8 wt % solution of crosslinked polyacrylamide hydrogel with a high molecular weight of approximately 10×10⁶ Dalton comprised of 3-dimensional polyacrylamide and purified water with a repeating unit of [—CH₂CH(CONH₂)—]ₙ, and each of the hydrogels described in the '348 patent.

Novel Features of bPAAGs

A very recent study has established that, rather than acting as a free-floating lubricant, the Noltrex Vet® hydrogel preferentially binds to damaged cartilage, aggregating to such cartilage, and forming a protective and lubricating film over such damage. Vishwanath, K, McClure, S. R., & Bonassar, L. J., Polyacrylamide Hydrogel Lubricates Cartilage After Biochemical Degradation And Mechanical Injury, J. ORTHOPAEDIC RESEARCH 1-9 (2022) (hereinafter “Vishwanath 2022”). The inventions herein described utilize the bPAAGs in new applications beyond their known use as a synovial fluid substitute.

### SUMMARY OF THE INVENTION

The present invention encompasses the prevention and treatment of various conditions with one or more bPAAGs.

In one embodiment, the invention relates to the method of treating damaged cartilage by administering to a human or animal a therapeutically effective amount of one or more bPAAGs. At least one of the bPAAGs may include, as a non-limiting example, a crosslinked polyacrylamide hydrogel product having molecular weight of approximately 10×106 Dalton and comprised of 3-dimensional polyacrylamide and purified water with a repeating unit of [—CH2-CH(CONH2)-]ₙ. As another non-limiting example, at least one of the bPAAGs may further comprise silver ions and/or a phosphate buffer. In some embodiments, the human or animal may have an allergy, sensitivity, or other adverse medication reaction to HA or CMS. In another embodiment, the bPAAGs may be combined with one or more therapeutics, such as antibodies, antifungals, and anti-inflammatory medicines to form an extended release composition.

The invention also relates to a method of prophylactically treating cartilage degeneration by administering to a human or animal a therapeutically effective amount of one or more bPAAGs. At least one of the bPAAGs may include, as a non-limiting example, a crosslinked polyacrylamide hydrogel product having molecular weight of approximately 10×106 Dalton and comprised of 3-dimensional polyacrylamide and purified water with a repeating unit of [—CH2-CH(CONH2)-]ₙ. As another non-limiting example, at least one of the bPAAGs may further comprise silver ions and/or a phosphate buffer. In some embodiments, the cartilage degeneration may be caused in whole or in part by osteoarthritis or, alternatively, a degenerative joint disease. In one embodiment, the bPAAGs re combined with anti-inflammatory medicines to form an extended release composition.

Another aspect of the invention includes the method of treating exposed collagen by administering to a human or animal a therapeutically effective amount of one or more bPAAGs. At least one of the bPAAGs may include, as a non-limiting example, a crosslinked polyacrylamide hydrogel product having molecular weight of approximately 10×10⁶ Dalton and comprised of 3-dimensional polyacrylamide and purified water with a repeating unit of [—CH2-CH(CONH2)-]ₙ. As another non-limiting example, at least one of the bPAAGs may further comprise silver ions and/or a phosphate buffer. In some embodiments, the human or animal may have an allergy, sensitivity, or other adverse medication reaction to HA or CMS. In another embodiment, the bPAAGs may be combined with one or more therapeutics, such as antibodies, antifungals, and anti-inflammatory medicines to form an extended release composition.

Other applications of bPAAGs that are covered by the present invention include methods for treating corneal ulcers; promoting wound healing, including for the treatment for burns; prohibiting tissue adhesions; acting as a localized and long-acting post-operative lubrication following joint reconstruction, arthroscopy, and other procedures that affect the synovial joints. The invention further includes the use of the bPAAGs polyacrylamide gel formulations as a carrier for controlled release of therapeutic agents, including but not limited to agents that should remain localized, as well as the use of bPAAGs as an alternative to CMC in scaffolds for tissue engineering and cell therapies, and as a lubricating substance.

### BRIEF DESCRIPTION OF THE DRAWINGS

FIG. 1, from Vishwanath 2022, illustrates the preferential binding to injured cartilage associated with the NoltrexVet® product, denoted in the article as pAAm. FIG. 1 depicts Safranin-O/Fast Green-stained histological sections of cartilage explants from an impact injury model. Clockwise, Block A shows healthy (Unimpacted) cartilage explants with PBC, Block B shows injured (Impacted) cartilage explants with PBC, Block C shows healthy (Unimpacted) cartilage explants treated with the Noltrex Vet® product, and Block D shows injured (Impacted) cartilage explants treated with the Noltrex Vet® product.

FIG. 2, also from Vishwanath 2022, illustrates another example of the preferential binding to injured cartilage associated with the NoltrexVet® product. FIG. 2 is second harmonic generation confocal images of cartilage explants incubated in fluorescently labeled polyacrylamide. FIG. 2 includes a control (Block A), biochemically degraded cartilage explant (IL-1β) (Block B), unimpacted cartilage explant (Block C), and impacted cartilage explant (Block D), all treated with the Noltrex Vet® product, denoted as pAAm. Increased fluorescence is representative of the localization of the NoltrexVet® product.

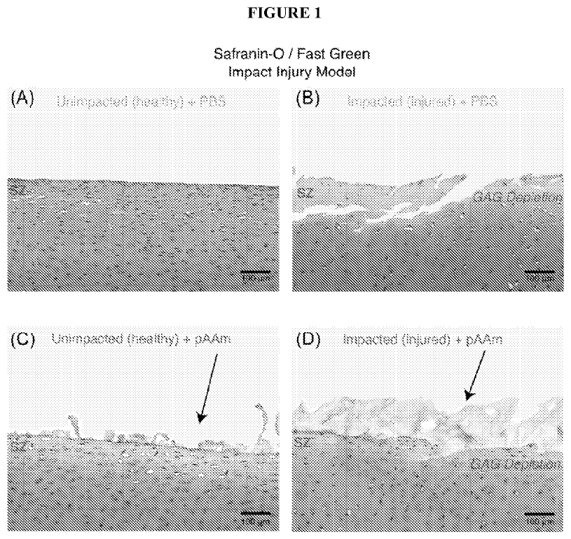

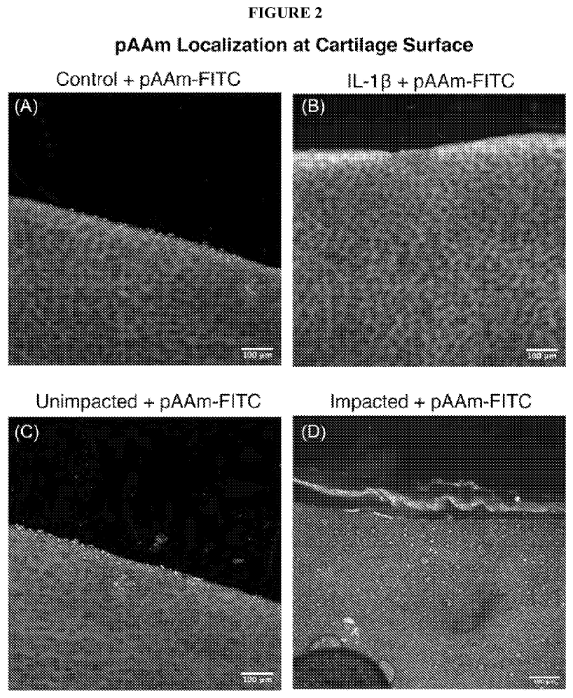

FIG. 1 and FIG. 2 cannot be adequately represented in black and white and thus are included in this application in color. The color figures may be viewed in Viswanath 2022.

### DETAILED DESCRIPTION OF EMBODIMENTS

The present invention relates to novel treatments and prophylaxis using the Noltrex Vet® product and other bPAAGs, the substitution of bPAAGs for HA and CMC in medical and veterinary applications, and the use of bPAAGs as a carrier for controlled release of therapeutic agents. As indicated in FIG. 1 and FIG. 2, the Noltrex Vet® product preferentially binds to areas of cartilage damage and exposed collagen.

In clinical patients presenting with corneal ulceration, the condition is diagnosed by the retention of fluorescein stain on the ocular surface. In one preferred embodiment, the Noltrex Vet® product and/or other bPAAGs are applied as a topical ophthalmic solution for the treatment of corneal ulcers. Such bPAAGs localize to the area of damaged tissue to provide a mechanical barrier. The barrier formed by bPAAGs would minimize exposure of the damaged tissue and allow for re-epithelialization of the cornea to occur. In another preferred embodiment, the bPAAGs may be combined with other therapeutics, including but not limited to antibiotics and antifungals. In one preferred embodiment, the therapeutics may be used as part of a bPAAG's extended release system, providing for an extended therapeutic release to occur over days to weeks. Use of therapeutics in a bPAAG extended release system may provide longer exposure to the products and/or more directed treatment of the damaged area. In one preferred embodiment, the administration of therapeutics using the extended release vehicle of bPAAGs will result in less frequent administration of the therapeutics required to treat corneal ulcers.

The affinity for accumulation of the bPAAGs in areas of corneal damage may be tested using fluorescently labelled bPAAGs in an ex-vivo model. In-vitro culture experimentation should confirm compatibility of the bPAAGs with corneal cells. Clinical efficacy may be determined through in-vivo applications using laboratory animals with induced corneal ulceration, and/or clinical patients presenting with corneal ulceration.

One non-limiting model for induced corneal ulceration would consist of the surgical creation of a superficial corneal ulcer. Amounts of approximately 0.1-0.2 ml of a bPAAG would be administered topically to the affected ocular surface, with greater volume possibly required with larger lesions. Foggy or blurred vision is expected following application, as is consistent with the use of lubricants. If prolonged vision impairment occurs, removal of free gel from the eye (that has not adhered to the ulceration), can be facilitated by lightly flushing with sterile water. Treatment would be continued until the corneal ulcer no longer retains fluorescein stain. Success of treatment typically would be measured by rate of wound healing, and presence/rate of adverse events, compared to standard treatment.

In another preferred embodiment, the Noltrex Vet® product and/or other bPAAGs are applied topically as a wound gel or applied in oral formulations for wounds and/or lesions within the gastric system. Indications include, but are not limited to, the use of bPAAGs for pressure ulcers, burn wounds, traumatic partial to full thickness injuries, and surgical partial to full thickness wounds. In some preferred embodiments, bPAAGs are used on wounds and/or lesions that heal by second intention through granulation and scar tissue formation as opposed to surgical closure. In other preferred embodiments, bPAAGs are used on wounds and/or lesions that are failing to heal properly, including as non-limiting examples, diabetic wounds and, in animals, exuberant granulation tissue and poorly vascularized decubital wounds. Additional preferred embodiments include the use of bPAAGs to treat gastric system wounds and/or lesions, including but are not limited to, gastric ulcers and/or gastrointestinal perforations. In the case of gastric lesions, the gel formulation would facilitate application through endoscope at site of the lesion, permitting treatment without precise application to the lesions given that the bPAAGs localize and attach to wound areas.

In treating wounds and/or lesions, the bPAAGs may be combined with other therapeutics, including but not limited to antibiotics, antifungals, anti-inflammatory medicines, and/or other compounds traditionally used to prevent infection and aid healing. In one preferred embodiment, the therapeutics may be used as part of a bPAAGs extended release system, providing for an extended therapeutic release to occur over days to weeks. Use of therapeutics in a bPAAG extended release system may provide longer exposure to the therapeutic products. In one preferred embodiment, the administration of therapeutics using the extended release vehicle of bPAAGs will result in less frequent administration of the therapeutics required to treat wounds and/or lesions.

The treatment of wounds and/or lesions, including burns, may also preferentially include the use of bPAAGs as a scaffold for the development of new cells, tissue engineering, treatments using stem cell and/or auto-grafts, and other scaffold-mediated treatments. Tissue scaffolds, also called extra-cellular matrices, are supporting structures composed of biocompatible materials that enable cell attachment and migration in tissue engineering. In some preferred embodiments, the bPAAG scaffold for wound treatment may be used in patients with allergies or sensitivity to CMC. Polyacrylamide gels have low allergenic properties; their use is even permitted for ingestible products, such as for a wash applied commercially to fruits and vegetables. The bPAAG scaffold may also have improved outcomes due to its unique affinity to preferentially localize to the site of injured tissue in-vivo providing a “homing mechanism” based on adhesion to exposed cartilage.

The affinity for accumulation of the bPAAGs to treat wounds may be tested using fluorescently labelled bPAAGs in an ex-vivo model. In a non-limiting example of a model, the target tissues would be exposed to the fluorescently labeled bPAAGs. The tissues would then be rinsed to remove non-adhered gel and imaged by, e.g., IVIS imaging, to confirm presence of the gel at the site of exposed collagen (site of lesion). Histopathology may also be used to additionally confirm the presence of bPAAG on the lesion of the investigated tissue.

In-vitro culture experimentation should confirm compatibility of the bPAAGs with wounds, tendons and/or internal organs for which the product may be used in lieu of HA or CMC for respective tissues. As one example of a model to confirm such utility, tissue cells would be cultured with the bPAAG in a laboratory setting. Biocompatibility would then be measured, which is determined by the amount of cellular proliferation, with multiple methods for measurement available. Cytotoxicity also would be measured, which can be performed by multiple methods known in the art, including, but not limited to, quantifying the number of alive versus dead cells as well as analysis using specific assays.

Clinical efficacy may be determined through in-vivo applications using laboratory animals with induced wounds and/or lesions, and/or clinical patients presenting with wounds and/or lesions, including but not limited to wounds and/or lesions that are failing to heal properly. An example of an animal model may include surgically creating a tendon lesion, and then comparing healing outcomes in those subsequently treated with the bPAAGs at the injury site versus controls. Analysis includes range of motion and gliding coefficients of the injured limb, as well as histological scoring for adhesions. Clinical efficacy would further be investigated in patients presenting with tendon injuries that are susceptible to adhesion formation. This intervention would need to be a first-line treatment, as the primary goal would be to prevent adhesion formation. The bPAAG would be a single injection, administered peritendinous, or in the tendon sheath as indicated by injury location, under aseptic technique. Dosing is expected to range between 0.5 to 6.0 ml, dependent on the anatomical location of the lesion, but may be lower or higher. Outcomes are determined by range of motion, and diagnostic imaging to assess soft tissue healing and the presence or absence of adhesion formation.

In a further preferred embodiment, bPAAGs are used to prevent tissue adhesions. In one preferred example, bPAAGs may be applied to tendon injuries to provide lubrication between the damaged tendon and surrounding tissues to prevent adhesions. The use of bPAAGs to prevent adhesions in tendon injury may be part of the treatment regimen of tendonitis and/or tendon fiber disruption or the sole treatment for such condition. Administration of bPAAGs to the damaged tendon may be non-invasive, such as by injection, and/or be used as part of prophylactic surgical intervention to prevent adhesions. In an additional alternative example, bPAAGs may be used in lieu of CMC to prevent adhesions that may form as a result of surgery, including but not limited to, abdominal surgeries.

The clinical efficacy of bPAAGs to prevent tissue adhesions may be determined through in-vivo applications using laboratory animals with tendon injury, and/or clinical patients presenting with tendon injuries.

Studies of the use of bPAAGs in abdominal surgeries may also be performed. Laboratory animal models may be used to determine efficacy in the prevention of intra-abdominal adhesion formation. In one such non-limiting model, an irritant would be administered into the abdomen, along with the bPAAG. After two weeks, the animals would be sacrificed and the adhesions would be macro- and microscopically graded.

Clinical efficacy may also be further investigated in patients undergoing abdominal surgery. Administration into the abdominal cavity would be performed at time of surgery. Dosing would be expected to range between 6 ml to 500 ml dependent on the size of the abdominal cavity. Outcomes would be determined based on development of symptoms that are consistent with adhesion formation, and a scheduled second-look laparoscopy to visualize the extent or absence of adhesions.

In another preferred embodiment, bPAAGs are used in synovial fluids affected by surgery. For example, arthroscopy, joint reconstruction, and joint replacement are surgical procedures where the use of bPAAGs may improve outcome. In certain surgeries, the synovial fluid is diluted and flushed and the body may not produce sufficient amounts of replacement fluid or do so in an expedient manner. The use of HA does not meet the needs in such surgeries, having a short lifespan and being subject to enzymatic degradation and migration. This condition has severe consequences in joint reconstruction using implants; the failure of adequate lubrication can lead to wear of the implants and/or autoimmune responses and conditions such as osteoarthritis. Use of bPAAGs to replace and enhance synovial fluid surrounding joint implants and prevent wear to the implant and the accumulation of particles on the implant surface are a preferred embodiment of the invention. In some preferred embodiments, bPAAGs also may be used as part of surgeries involving synovial joints as a post-surgical lavage and/or as an injection following surgery. As an example of a post-surgical lavage, bPAAGs would be injected at the end of the irrigation process during surgery to wash out the remaining irrigation solution, and restore lubrication to the joint surfaces.

Clinical efficacy may be determined through in-vivo applications using clinical patients presenting with synovial injuries requiring surgical interventions. For example, patients undergoing arthroscopic surgery may be randomly assigned to the treatment group, or to the control group. Outcomes following surgery to measure would include comfort/pain level, joint swelling and function, return to function timeline, and number of adverse events. Trials where post-surgical lavage using bPAAGs occurs, as well as the use of bPAAGs as lubrication following joint replacement with implants may also be investigated.

The present invention also includes a preferred embodiment whereby bPAAGs are used as a prophylactic treatment for OA and degenerative joint disease (DJD) more broadly. In some preferred examples, bPAAGs may be administered in specific joints that are at higher risk of developing OA or DJD. In other preferred examples, bPAAGs may be administered in joints that have conformational irregularities, joint angles, and/or where there is injury to surrounding tissues of the joint that puts such joints at a higher risk of developing OA or DJD. In some preferred embodiments, bPAAGs may be used in athletes in joints that are differentially strained because of the sport or competition in which the athlete is engaged. For example, one animal athlete that carries high risk of preferential joint damage and inflammation is the thoroughbred racehorse, with the metacarpophalageal joint associated with almost 50% of all joint related problems impacting ability to race. In other preferred embodiments, bPAAGs may be used for those who perform repetitive tasks where such an application would act to help prevent degradation of cartilage.

In some preferred embodiments of the present invention, bPAAGs may be used as a prophylactic treatment to slow or prevent degradation (or further degradation) of cartilage. In one such preferred applications, bPAAGs may be applied as part of the healing process of a traumatic injury to cartilage as a method to prevent further damage. In other preferred applications, bPAAGs may be applied after healing of traumatic injury or surgery relating to damaged cartilage. In another preferred application, bPAAGs may be applied in anticipation of a particular stress event that may result in further injury as part of preparation for undertaking a high-risk or high-strain athletic event. This application would provide physical protection to the cartilage surface prior to undergoing excessive movement that causes pain, inflammation, and affects normal movement. In humans, one example may be the use of bPAAG in a knee prior to football training camp. In animals, one example may include the use in a racehorse metacarpophalangeal joint prior to training season.

The prophylactic abilities of bPAAGs to prevent degradation (or further degradation) or slow the degradation process may be evaluated using known animal experimental degenerative models where injection of the bPAAGs is compared against control. One non-limiting animal model of evaluating prophylactic abilities of bPAAGs would use horses is a 70-day cartilage groove model. In this model, surgical injury is applied to the articular cartilage of the metacarpophalangeal joints followed by a set exercise program. Treatment (a set amount of bPAAG, such as, but not limited to, 2.5 ml) would be administered intra-articular two weeks following the surgical procedure, and outcomes would compared to saline control. Efficacy of treatment would be determined based on radiographic changes, histologic changes, cartilage turn-over markers, and severity of symptoms/pain. In this model, the horses enrolled are adult horses with no pre-existing conditions that would limit their participation. A total of 13 horses would required to obtain outcomes of 95% confidence level with 80% power.

Clinical studies also may be undertaken to evaluate the prophylactic effects of bPAAGs on joint damage. To determine clinical efficacy in humans, the study would require demonstration of disease and the measurement of symptom modification. Human clinical trials would include individuals with mild to moderate knee arthritis. Treatment would consist of intra-articular injection of a set volume of bPAAGs, such as, but not limited to, 4 ml. The frequency of the treatment for the patients of the study would influenced by underlying reason for treatment, i.e., athletic performance, conformational abnormality, injury that affects joint stability, etc. Because of the known long-lasting effects of bPAAGs, frequency may be as little as once every six months to once every two or more years. Outcomes of clinical studies would be measured in compliance with FDA requirements for disease-modification osteoarthritis treatment, where cartilage thickness is determined by diagnostic imaging, and pain scoring informs on symptom modification abilities.

Additional preferred uses for bPAAGs include its use as a controlled release agent. While discussed above in particular contexts, bPAAGs may be combined with any compatible therapeutic to cause the slow release of such therapeutics for the treatment of any disease or condition. In some embodiments, bPAAGs may be chemically bound to conventional therapeutics. In some embodiments, one or more conventional therapeutics may be bound to bPAAGs by hydrogen-binding or functional hydroxy groups, providing extended release lasting for weeks to months. In some embodiments of the present invention, one therapeutic may be used with a single bPAAG to provide for controlled release of the therapeutic.

bPAAGs are not subject to enzymatic degradation. The release of the therapeutic from the bPAAGs occurs via a surface degradation system, as the polymer undergoes macrophage resorption, a process known to take weeks to months depending on the localization of the injection. This rate of degradation may be adjusted based on, e.g., the chemical composition of the bPAAG and its crosslinking density.

In some embodiments, one therapeutic may be used with two or more different bPAAGs for controlled release where the different bPAAGs allow for the release of the therapeutic at two different rates. For example, a therapeutic may be released from a bPAAG with a faster rate of degradation to provide short term release as well as from a bPAAG with a slower rate of degradation to provide for release lasting an extended period of time.

In other embodiments, a controlled release product may be made to administer two or more therapeutics each with at their own rate; doing so by binding each therapeutic to a different bPAAG having the desired kinetic properties.

Therapeutic agents that are particularly beneficial as an extended-release formulation include, but are not limited to, growth factors, antimicrobials, and antineoplastic agents, and anti-inflammatories. Tissues where these formulations would be beneficial include synovial structures, peri-tendinous, cornea, and other tissue injuries with exposed collagen. Growth factors aid in tissue healing, and localization to the site of injury with slow release allows for prolonged therapeutic value. Antimicrobials can provide prolonged therapeutic value in infections that are difficult to treat, for example hospital acquired MRSA infection following joint surgery. The combination of antimicrobials or anti-inflammatories with the bPAAG as an extended-release formulation could also be used in corneal ulceration treatment to decrease the frequency in which application of these products are required. As further preferred embodiments, bPAAGs may be used as a controlled release vehicle in tissues that are irritated by traditional oil-based carrier substances, including but not limited to synovial structures and the cornea. Extended release agents are often not available for these tissues due to their irritability, and thus requires multiple drug administrations or there may be no suitable drug options due to an inability to maintain therapeutic levels.

In another preferred embodiment of the present invention, bPAAGs may be used in combination with biologic therapies to act as a matrix or scaffold material helping to further localize the effects of these products. In some preferred example, bPAAGs may be used as a scaffold for the application of stem cells, transplanted cells or tissues, proteins, or other biologic components. In one application, growth factors may be added to a bPAAG scaffold to facilitate long-term support. In another application, bPAAGs could be used as a medium to culture stem cells in a laboratory setting. The affinity of bPAAGs to localize in areas of exposed collagen improves localization of the stem cells to a site of injury. In this example, cultured cartilage stem cells could have better localization to the area of cartilage damage, as opposed to use of a carrier without this characteristic.

The present invention also includes the preferred use of bPAAGs as an alternative to HA and/or CMC. Preferably, bPAAGs are used as a substitute for CMC in applications including but not limited to wound gels, scaffold in tissue engineering, ophthalmic solutions, drug carriers and extended release formulations, and/or tissue adhesions. In other preferred embodiments, bPAAGs are used as a substitute for HA in applications including but not limited to viscosupplementation and reduction of soft tissue adhesions in synovial structures. In preferred examples of the invention, bPAAGs are used as a substitute for CMC and/or HA in mammals and humans that are allergic or sensitive to CMC and/or HA. The bPAAG's interaction with tissue surfaces provides a distinct advantage for localization of therapeutic effect, unlike CMC or HA. As a synthetic polymer, allergic reactions are rare to bPAAGs as well. CMC and HA allergies are more common due to cellulose and protein components, respectively.

In the treatments and products described above, bPAAGs also may be combined with any compatible excipients and/or with other therapeutic agents.

Administration of bPAAGs may be by any known technique for the particular treatment or prophylaxis described herein, including but not limited to, administration that is topical, oral, by lavage, or by injection.

The therapeutically effective amount of bPAAGs also will vary depending upon the method of treatment or prophylaxis, the manner of administration, the kind and size of the human or animal, the bPAAG used, and/or the severity of the condition, etc. Below is a chart of exemplary therapeutic amounts for various claimed treatments of the present invention:

CHART 2

| Treatments | Dosage amounts |
|---|---|
| Intra-articular, bursitis, | 0.5 ml to 6 ml |
| tendon adhesion | |
| Corneal ulcers | 0.1 ml to 1.0 ml |
| (ulcerative keratitis) | |
| Topical Wound | 0.5 ml/cm² to 1.0 ml/cm² |
| Lavage: post-surgical, | 6 ml to 500 ml |
| intra-abdominal | |
| Extended-release | 1 ml to 20 ml |
| injectable therapeutics | |
| Injectable scaffold | 0.5 ml/cm² to 1.0 ml/cm² |

The amounts provided for various treatments in Chart 2 are not to be considered minimum or maximum amounts given that the effective therapeutic amount may be higher or lower based on a number of factors. Factors that might commonly affect dosage amounts includes size and depth of wound or injury to tissue and anatomical size of the structure being treated. Although the present inventions and their advantages have been described in detail, it should be understood that various changes, substitutions and alterations can be made herein without departing from the spirit and scope of the disclosure as defined by the appended claims. Moreover, the scope of the present application is not intended to be limited to the particular compositions of matter, means, methods and steps described in the specification. As one of ordinary skill in the art will readily appreciate from the present disclosure, the compositions of matter, means, methods, or steps, presently existing or later to be developed that perform substantially the same function or achieve substantially the same result as the corresponding examples described herein may be utilized according to the present disclosure. Accordingly, the appended claims are intended to include within their scope such compositions of matter, means, methods, or steps.

1. A method of treating damaged cartilage in a human or animal in need thereof, the method comprising administering to the human or animal a therapeutically effective amount of one or more bPAAGs.

2. The method of claim 1, wherein at least one of the bPAAGs is a crosslinked polyacrylamide hydrogel product having molecular weight of approximately 10×10⁶ Dalton and comprised of 3-dimensional polyacrylamide and purified water with a repeating unit of [—CH₂—CH(CONH₂)—]ₙ.

3. The method of claim 2, wherein the at least one bPAAG further comprises silver ions, a phosphate buffer, or both silver ions and a phosphate buffer.

4. The method of claim 1, wherein the human or animal in need thereof further has an allergy, sensitivity, or adverse medical reaction to HA or CMC.

5. (canceled)

6. The method of claim 3, wherein the human or animal in need thereof further has an allergy, sensitivity, or adverse medical reaction to HA or CMC.

7. The method of claim 1, wherein the one or more bPAAGs are further combined with one or more therapeutics selected from the group of antibiotics, antifungals, and anti-inflammatory medicines to form an extended release composition.

8. (canceled)

9. The method of claim 3, wherein the one or more bPAAGs are further combined with one or more therapeutics selected from the group of antibiotics, antifungals, and anti-inflammatory medicines to form an extended release composition.

10. A method of prophylactically treating cartilage degeneration in a human or animal in need thereof, the method comprising administering to the human or animal a therapeutically effective amount of one or more bPAAGs.

11. The method of claim 10, wherein at least one of the bPAAGs is a crosslinked polyacrylamide hydrogel product having molecular weight of approximately 10×10⁶ Dalton and comprised of 3-dimensional polyacrylamide and purified water with a repeating unit of [—CH₂—CH(CONH₂)—]ₙ.

12. The method of claim 11, wherein the at least one bPAAG further comprises silver ions, a phosphate buffer, or both silver ions and a phosphate buffer.

13. (canceled)

14. (canceled)

15. The method of claim 12, wherein the cartilage degeneration is caused in whole or in part by osteoarthritis or a degenerative joint disease.

16. The method of claim 10, wherein the one or more bPAAGs are further combined with anti-inflammatory medicines to form an extended release composition.

17. (canceled)

18. The method of claim 12, wherein the one or more bPAAGs are further combined with anti-inflammatory medicines to form an extended release composition.

19. A method of treating exposed collagen in a human or animal in need thereof, the method comprising administering to the human or animal a therapeutically effective amount of one or more bPAAGs.

20. The method of claim 19, wherein at least one of the bPAAGs is a crosslinked polyacrylamide hydrogel product having molecular weight of approximately 10×10⁶ Dalton and comprised of 3-dimensional polyacrylamide and purified water with a repeating unit of [—CH₂—CH(CONH₂)—]ₙ.

21. The method of claim 20, wherein the at least one bPAAG further comprises silver ions, a phosphate buffer, or both silver ions and a phosphate buffer.

22. The method of claim 19, wherein the human or animal in need thereof further has an allergy, sensitivity, or adverse medical reaction to HA or CMC.

23. (canceled)

24. The method of claim 21, wherein the human or animal in need thereof further has an allergy, sensitivity, or adverse medical reaction to HA or CMC.

25. The method of claim 19, wherein the one or more bPAAGs are further combined with one or more therapeutics selected from the group of antibiotics, antifungals, and anti-inflammatory medicines to form an extended release composition.

26. (canceled)

27. The method of claim 21, wherein the one or more bPAAGs are further combined with one or more therapeutics selected from the group of antibiotics, antifungals, and anti-inflammatory medicines to form an extended release composition.
